(19) United States
(12) Patent Application Publication
(10) Pub. No.: US 2023/0363714 A1
(43) Pub. Date: Nov. 16, 2023

(54) Device for Measurement of Core Body Temperature and Temperature Monitoring Methods

(71) Applicant: University of Pittsburgh - Of the Commonwealth System of Higher Education, Pittsburgh, PA (US)

(72) Inventors: Deirdre Nolfi-Donegan, Pittsburgh, PA (US); Michael Landau, Pittsburgh, PA (US); Quyen Nguyen, Pittsburgh, PA (US)

(21) Appl. No.: 18/248,145

(22) PCT Filed: Oct. 7, 2021
(86) PCT No.: PCT/US2021/053913

§ 371 Date: Apr. 6, 2023

Related U.S. Application Data
(60) Provisional application No. 63/089,279, filed on Oct. 8, 2020.

(51) Int. Cl. A61B 5/00; A61B 5/01

(52) U.S. Cl. CPC A61B 5/686; A61B 5/0022; A61B 5/01; A61B 5/0008; A61M 39/0208

(57) ABSTRACT

A temperature measurement device configured to be attached to an implantable medical device for measuring a core body temperature of a patient includes a housing having a top, a bottom, and at least one sidewall extending between the top and the bottom of the housing enclosing an interior. The device also includes: a temperature sensor connected to the housing and/or enclosed within the interior of the housing; a wireless transmitter enclosed within the interior of the housing; and at least one processor enclosed within the interior of the housing electrically connected to the temperature sensor and to the wireless transmitter. The at least one processor is configured to: receive and process data representative of a core body temperature of the patient from the temperature sensor; and cause the wireless transmitter to transmit the processed data to a remote device or computer network.

### CROSS REFERENCE TO RELATED APPLICATION

The present application claims the benefit of U.S. Provisional Patent Application No. 63/089,279, filed Oct. 8, 2020, the disclosure of which is hereby incorporated by reference in its entirety.

### BACKGROUND OF THE INVENTION

### Field of the Invention

The present disclosure is directed to devices and methods for measurement of core body temperature of a patient and, in particular, to a temperature measurement device that is mounted, adhered, connected to, or integrated within an implantable medical device, such as an access port and/or patient central line, and which wirelessly transmits a patient's core body temperature to a remote electronic device or computer network.

### Description of Related Art

Patients undergoing chemotherapy treatment or treatment with other cancer-fighting medicines, IV treatments (e.g., intravenous infusion of antibiotics and nutrition), and/or hemodialysis treatment for kidney failure are often provided with a central line or venous catheter. The central line or venous catheter can be used, for example, to provide a drug infusion, blood infusion, or for providing nutrition to the patient. The central line or venous catheter can be accessed through an implanted venous access port (often referred to as a “mediport” or a “port”), which allows for infusion of medications and nutrition for the patient through the port to the central line or venous catheter. Providing substances to the patient's vasculature through the port and venous catheter is generally believed to be preferred compared to puncturing a vein to provide venous access every time such access is needed. Unfortunately, implanted medical ports and venous catheters or central lines can be an infection risk, in particular, creating a risk that a patient will develop a serious bloodstream infection. Progression of the blood stream infection can cause the patient to develop a fever (for example, an elevated body temperature in excess of 38° C. for humans), which is a symptom of a systemic infection. For an immune-comprised patient, a fever can be a medical emergency, requiring quick recognition and response, such as admission to an emergency room or another medical facility. Patients with implanted long-term venous catheters or central lines are often discharged from a hospital and spend substantial amounts of time at home, meaning that the patient and/or the patient's caregiver must monitor the patient for signs of infection, fever, and respond appropriately.

For many patients, home-based fever detection is limited to manually checking the patient's body temperature using, for example, an oral or skin-contact thermometer, or other wearable external temperature-monitoring device or equipment as are known in the art, which may not be accepted or easy to use for many patients. Further, deciding how and when to check a patient's temperature can be a source of stress and anxiety for caregivers, especially for those that care for pediatric, non-communicative, and/or elderly patients, such as patients in memory care facilities, who cannot express when they are feeling ill or if they believe that they are developing a fever. Concerns and anxieties about caring for non-communicative patients can cause decreased quality of life for both caregiver and patient,

Several currently available devices and methods exist for tracking a patient's core body temperature. However, most available devices and methods for monitoring core body temperature are not used in a home-care setting. Invasive devices and methods are available, such as implantable temperature probes that detect a temperature in a patient's artery or vein. For example, a temperature probe implanted in a patient's pulmonary artery (PA) can be used to provide a highly accurate core body temperature measurement. However, such invasive methods are usually only available for hospitalized patients and not for patients at home. Other temperature measurement devices and methods can include esophageal, rectal, and indwelling urinary catheter temperature probes. However, such devices may be contraindicated in cancer and other immune--compromised patients for infectious reasons. Also, such invasive probes may not be feasible for patients at home.

Ingestible temperature sensors, such as the CorTemp® Ingestible Thermometer Pill by HQ, Inc., can be used for obtaining temporary core temperature measurements for patients. However, such ingestible devices only provide temperature measurements for a short time (e.g., several hours) before being passed from the body. Therefore, in order to monitor a patient's core body temperature over a period of days or weeks, many temporary-use ingestible sensors would be needed. In addition, caregivers would be responsible for monitoring status of the sensors to ensure that appropriate temperature readings are being received from the ingested sensors. Accordingly, temporary ingestible sensors may not be a good option for monitoring patients at home over extended periods of time.

Effective devices and methods for real-time monitoring of a patient's core temperature that are amenable to home use and/or unskilled use, are needed.

### SUMMARY OF THE INVENTION

According to an aspect of the present disclosure, a temperature measurement device configured to be attached to an implantable medical device for measuring a core body temperature of a patient includes a housing having a top, a bottom, and at least one sidewall extending between the top and the bottom of the housing enclosing an interior. The device also includes: a temperature sensor connected to the housing and/or enclosed within the interior of the housing; a wireless transmitter enclosed within the interior of the housing; and at least one processor enclosed within the interior of the housing electrically connected to the temperature sensor and to the wireless transmitter. The at least one processor is configured to: receive and process data representative of a core body temperature of the patient from the temperature sensor; and cause the wireless transmitter to transmit the processed data to a remote device or computer network.

According to another aspect of the disclosure, an implantable assembly includes: the previously described temperature measurement device; and a venous access port connected to the temperature measurement device. The venous access port includes a housing having an open top, a closed bottom, a sidewall extending between the top and the bottom of the housing. The venous access port also includes a catheter port extending from the sidewall of the housing; a pierceable, self-sealing septum over the open top of the housing configured for injection of liquids into a fluid chamber enclosed by the housing; and a venous catheter connected to and extending from the catheter port. The venous catheter is configured to extend from the catheter port into a vein of the patient and is fluidly connected to a fluid chamber enclosed by the housing for moving fluid injected into the fluid chamber through the venous catheter and to the vein of the patient.

According to another aspect of the disclosure, an implantable medical device includes: a housing having an open top, a bottom, and at least one sidewall extending between the top and the bottom of the housing, and a catheter port extending from the sidewall of the housing. The housing defines a fluid chamber in fluid communication with the catheter port and a separate interior. The implantable medical device also includes a pierceable, self-sealing septum over the open top of the housing configured for injection of liquids into the fluid chamber of the housing; and a venous catheter connected to and extending from the catheter port. The venous catheter is configured to extend from the catheter port into a vein of the patient and is fluidly connected to a fluid chamber of the housing so that fluid injected into the fluid chamber passes through the venous catheter and to the vein of the patient. The implantable medical device also includes a temperature sensor connected to and/or enclosed within the interior of the housing; a wireless transmitter enclosed within the interior of the housing; and at least one processor enclosed within the interior of the housing electrically connected to the temperature sensor and to the wireless transmitter. The at least one processor is configured to: receive and process data representative of a core body temperature of the patient from the temperature sensor; and cause the wireless transmitter to transmit the processed data to a remote device or computer network.

According to another aspect of the disclosure, a temperature monitoring system includes: any of the previously described assemblies or devices; and at least one remote electronic device configured to directly or indirectly receive processed data from the temperature measurement device and output information about a core body temperature of a patient determined based on the received data.

According to another aspect of the disclosure, a method of monitoring a core body temperature of a non-communicative patient includes: attaching any of the previously described temperature measurement devices to an implantable medical device; implanting the :implantable medical device and attached temperature measurement device to the patient; and monitoring a remote electronic device in wireless communication with the temperature measurement device to determine if the patient has a fever (e.g., a core body temperature above about 38° C., 39° C., or 40° C.). The remote electronic device is configured to: receive the processed data from the temperature measurement device; and provide an indication of the core body temperature of the patient via the remote electronic device.

According to another aspect of the disclosure, use of any of the previously described devices or assemblies for monitoring a core body temperature of a non-communicative patient is provided. The patient is optionally undergoing at least one of chemotherapy treatment or treatment with other cancer-fighting medicines, IV treatments (e.g., intravenous infusion of antibiotics and nutrition and/or hemodialysis treatment for kidney failure.

According to another aspect of the disclosure, use of any of the previously described temperature measurement devices or assemblies for monitoring a core body temperature of a patient is provided. The patient is a child, an elderly patient, and/or a patient with dementia or Alzheimer's disease.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

### BRIEF DESCRIPTION OF THE DRAWINGS

FIG. 1A is a top view of a temperature measurement device, according to an example of the present disclosure;

FIG. 1B is a side view of the temperature measurement device of FIG. 1A;

FIG. 1C is a cross-sectional view of the temperature measurement device of FIG. 1A, taken along line 1C-1C;

FIG. 2A is a top view of a venous access port, as is known in the prior art;

FIG. 2B is a side view of the venous access port of FIG. 2A;

FIG. 2C is a cross-sectional view of the venous access port of FIG. 2A taken along line 2C-2C;

FIG. 3A is a side view of a temperature measurement device adhered to an implantable medical device, according to an aspect of the present disclosure;

FIG. 3B is a cross-sectional view of the temperature measurement device and implantable medical device of FIG. 3A taken along line 3B-3B;

FIG. 4 is a schematic drawing showing a temperature measurement device and implantable medical device implanted in a patient, according to an aspect of the present disclosure;

FIG. 5A is a schematic drawing of a temperature monitoring system including an implantable temperature measurement device, according to an aspect of the present disclosure;

FIG. 5B is a drawing of a user interface screen showing a patient's core body temperature, according to an aspect of the present disclosure;

FIG. 6 is a flow chart showing a method for monitoring a patient with an implantable temperature measurement device, according to an aspect of the present disclosure;

FIG. 7A is a side view of an integrated device including a temperature measurement device and a venous access port, according to an aspect of the present disclosure;

FIG. 7B is a cross-sectional view of the integrated device of FIG. 6A taken along line 7B-7B; and

FIG. 8 is a schematic drawing of a computer device or controller according to an aspect of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. it is also to be understood that the specific devices and processes illustrated in the attached drawings, and described in the following specification, are examples. Further, depicted elements are not necessarily to scale, but are depicted in a manner to facilitate the showing of any described element and its relation to other elements of a described device, Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about” Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements,

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising,” “comprise” or “comprised,” and variations thereof, are meant to be open ended.

As used herein, the term “patient” refers to members of the animal kingdom including but not limited to human beings.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

The present disclosure is directed to temperature measurement devices 10 and methods for measuring the core body temperature of a patient. As used herein, the “core body temperature” of a patient refers to a temperature measurement obtained from within a body cavity of the patient. Core body temperature can be obtained, for example, from a temperature sensor, probe, or device located within, for example, a subcutaneous space below the patient's skin, a hollow body space or cavity (e.g., the abdominal cavity), an organ (e.g., heart, lungs, kidney, intestines, rectum), or vasculature (e.g., arteries or veins). Core body temperature, is more accurate than external temperature measurements, such as measurements obtained in the mouth or by a temperature sensor in contact with the patient's skin.

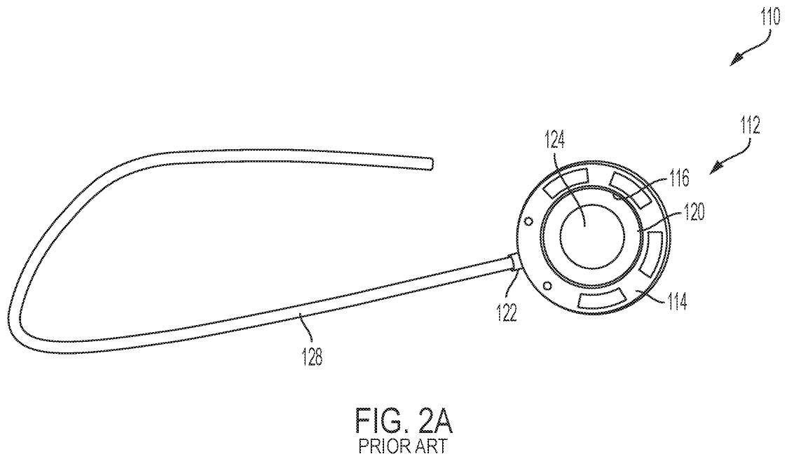

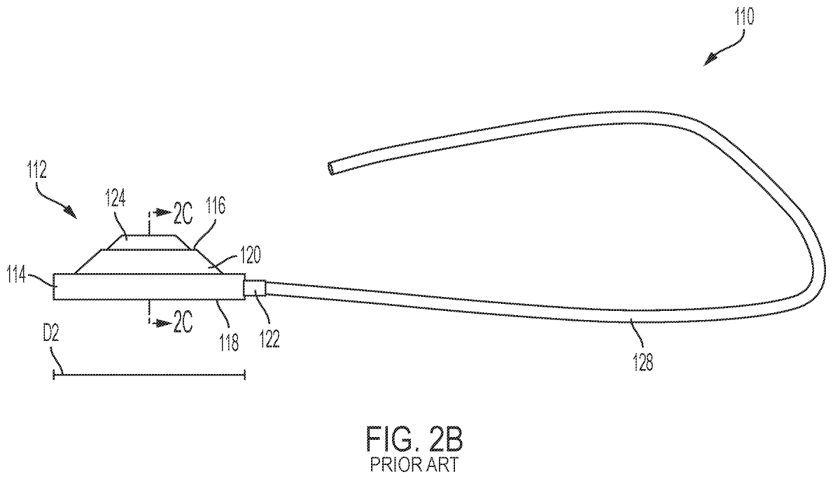

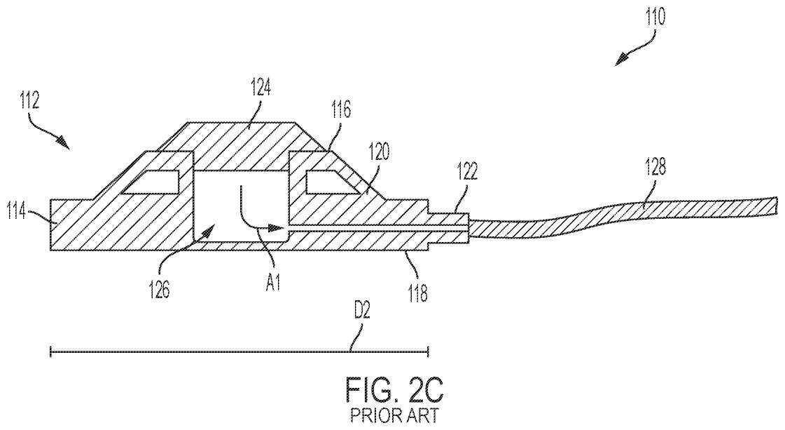

In some examples, the temperature measurement device 10 is configured to be connected, mounted, attached to, or integrated with an implantable medical device 110. For example, as exemplified in the device shown in FIGS. 2A-2C, the implanted medical device 110 can be an implantable cardiovascular device, such as an access device, port, drain, huh, or similar implantable device configured to be implanted in the patient's cardiothoracic region and/or in proximity to the patient's heart or lungs. In some examples, the implantable medical device 110 is, or can include, an implantable medical port, such as the previously described implantable venous access port 112 (e.g., a mediport). Non-limiting, commercial examples of implantable venous access ports 112 that can be used with the temperature measurement devices 10 of the present disclosure include, for example, the Port-ACath® implantable venous access system by Smiths Medical, the Ultra Slimport™ and PowerPort™ by Becton, Dickinson and Company, and the NorPort™ by Norfolk Medical.

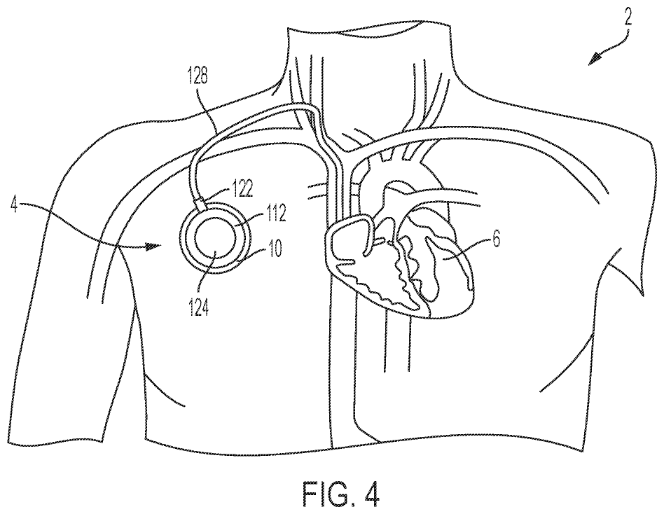

In some examples, the implantable venous access port 112 can include a body or port housing 114 having an open top 116, a flat closed bottom 118, a sidewall 120 extending between the top 116 and the bottom 118 of the housing 114. The housing 114 also includes at least one catheter port 122 extending from the sidewall 120 of the housing 114. The implantable venous access port 112 also includes a pierceable, self-sealing septum 124, such as a septum 124 formed from an elastomeric material, over the open top 116 of the housing 114 configured to be used for injection of liquids into a fluid chamber 126 enclosed by the housing 114. The implantable venous access port 114 also includes a venous catheter 128 connected to and extending from the catheter port 122. The venous catheter 128 can be configured to extend from the catheter port 122 into a vein of the patient. The venous catheter 128 is fluidly connected to a fluid chamber 126 (shown in FIG. 2C), so that fluid injected into the chamber 126 through the septum 124 flows into the catheter 128 and through the catheter 128 to the patient's' vein, as shown by arrow A1 in FIG. 2C. Venous access ports 112 are often implanted within a subcutaneous space of the patient. For example, as shown in FIG. 4, an implantable venous access port 112 and temperature measurement device 10 can be positioned under skin of a patient 2 and above muscle tissue and/or ribs. The port 112 can be positioned on a front portion of the patient's chest 4. The catheter 128 extends from the port 112 into a vein of the patient 2 and extends through the vein towards the patient's heart 6. In other examples, an implantable venous access port 112 and temperature measurement device 10 can be positioned in a forearm, upper arm, leg, or any other convenient body location of a patient 2 depending on which vein is being accessed.

In some examples, as described in further detail hereinafter, the temperature measurement device 10 of the present disclosure can be a self-contained, temperature-sensing device that attaches to an external posterior wall of the implantable venous access port 112 by a biocompatible adhesive. Further, in some examples, the temperature measurement device 10 comprises a temperature sensor including a thermistor. The temperature measurement device 10 can also include internal electronics, such as a wireless transmitter and a battery.

Unlike manual thermometers, the temperature measurement device 10 of the present disclosure can be configured to provide a continuous or periodically updated core body temperature measurement for the patient 2. This continuous or predictable periodic temperature measurement reduces or eliminates the need for caregivers, such as parents of pediatric patients, to, for example, wakeup multiple times throughout the night to manually check a patient's temperature. Accordingly, it is believed that the temperature measurement device 10 and associated temperature monitoring systems and methods of the present disclosure improve accuracy and consistency of temperature monitoring. It is believed that this improved accuracy and consistency provided by the temperature measurement device 10 of the present disclosure improves quality of life for caregivers of high-risk immune-comprised patients by removing burdens and anxieties related to fever surveillance. Further, the temperature measurement device 10 and temperature monitoring systems and methods of the present disclosure help caregivers to more consistently and accurately monitor body temperature for non-communicative patients, such as a child, an elderly patient, and/or a patient with dementia, Alzheimer's, or who otherwise cannot express when they are feeling ill or are developing a fever.

### Temperature Measurement Device

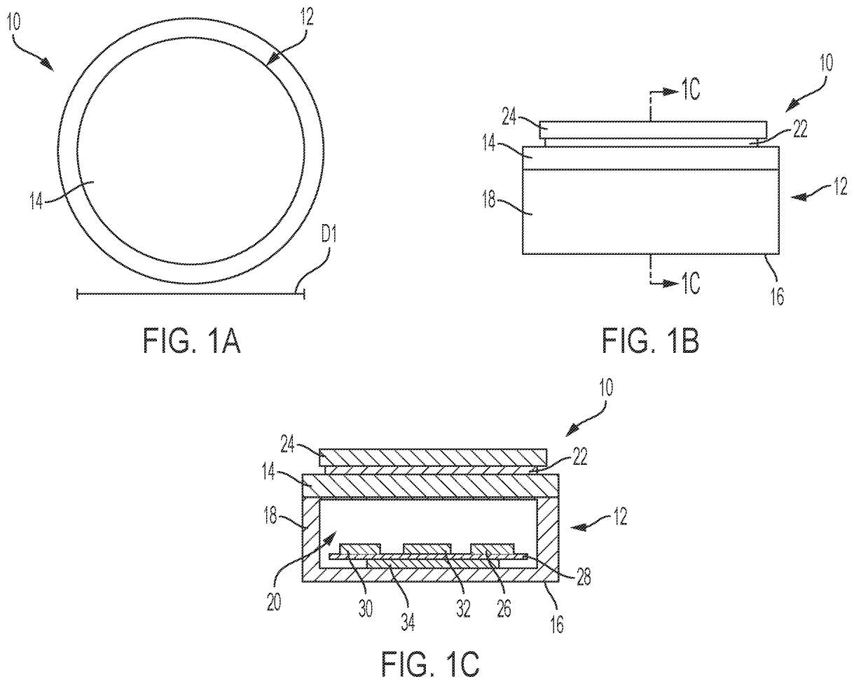

With reference to FIGS. 1A-5B, in some examples, the temperature measurement device 10 of the present disclosure includes a housing 12 having a top 14, a bottom 16, and sidewall 18 extending between the top 14 and the bottom 16 of the housing 12 enclosing an interior 20 of the housing 12. The housing 12 can be formed from any suitable material or combination of materials. The housing 12 can be formed from a rigid material, such as from a biocompatible hard plastic material, such as polyurethane or high-density polyethylene (HDPE). The housing 12, along with other structural and electronic components of the temperature measurement device 10, can be non-metallic, non-conductive, and/or MRI compatible. The interior 20 of the housing 12 can be any suitable shape and/or volume so long as it is consistent with functionality of the temperature measurement device 10. The volume of the interior may be ≥0 mm³ (no gap). Further, the interior 20 can be filled with any compatible substance, such as a gas (air, or an inert gas), an adhesive, or a filler, such as a polymeric filler, such as an epoxy resin.

The housing 12 can be any suitable size and shape. In some examples, the housing 12 can be a size and shape selected to correspond to a size and shape of the implantable medical device 110. For example, a size and shape of the top 14 or the bottom 16 of the temperature measurement device housing 12 can match and/or be substantially equivalent to a size and shape of a surface of the implantable medical device 110 to which the housing 12 is to be attached. Matching or being substantially equivalent to can mean that the size of the top surface 14 of the temperature measurement device 10 is the same as or slightly smaller than (e.g., within about 5%, 10%, or 25%) a size of the flat bottom surface 118 of the implantable medical device 110. For example, for a circular implantable venous access port 112 and circular temperature measurement device 10, a diameter D1 of the top 14 of the temperature measurement device 10 can be about the same (e.g., within 5%, 10%, or 25% of) as the outer diameter D2 (shown in FIGS. 2B and 2C) of the bottom 118 of the implantable medical device housing 114. For a port 112 and/or temperature measurement device 10 that is a square or rectangle shape, a height and/or length of the temperature measurement device 10 can be the same as or slightly smaller than (e.g., within about 5%, 10%, or 25%) a height and/or length of the venous access port 112.

In some examples, the temperature measurement device housing 12 can be formed from multiple parts or pieces that are connected together to enclose electronic components of the device 10. For example, the housing 12 can include a body or box portion and a top or lid. The top or lid can be placed over an open top of the body or box. Further, the parts of the housing 12 can be sealed together forming, for example, a permanent (e.g., non-removable) waterproof enclosure, thereby securely enclosing electrical components of the temperature measurement device 10 within the housing 12.

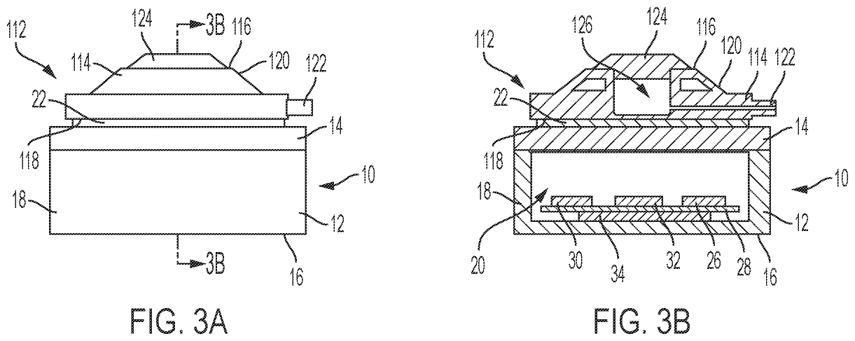

As previously described, the temperature measurement device 10 can be configured to be connected, mounted, or adhered to an implantable medical device, e.g., the implantable medical device 110, as shown in FIGS. 3A and 3B. In order to attach the temperature measurement device 10 to the implantable medical device 110, the temperature measurement device 10 can include an adhesive layer 22 over the top 14 and/or the bottom 16 of the device housing 12 for attaching the housing 12 to a surface of the implantable medical device 110. As shown in FIGS. 1B and 1C, the adhesive layer 22 can be coated on or over all or a portion of the top 14 of the housing 12 for adhering the top 14 of the housing 12 to, for example, the flat bottom surface 118 of the implantable venous access port 112. The temperature measurement device 10 can also include a removable cover sheet 24, such as a disposable paper cover sheet. over the adhesive layer 22 prior to use of the temperature measurement device 10. The cover sheet 24 can be removably attached to the adhesive layer 22 and configured to be removed to expose the adhesive layer 22. Once the adhesive layer 22 is exposed, the temperature measurement device 10 can be adhered to the venous access port 112. In other examples, the temperature measurement device 10 can include different types of mechanical connectors or fasteners, such as clips, protrusions, barbs, or other connectors, as are known in the art, for securing the temperature measurement device 10 to the venous access port 112.

In some examples, the temperature measurement device 10 further includes a temperature sensor 26 connected to the housing 12 and/or enclosed within the interior 20 of the housing 12. For example, the temperature sensor 26 can be a computer chip (e.g., a silicon chip comprising a temperature-sensitive material having temperature dependent electrical properties) or a micro- or nano-electromechanical systems (MEMS/NEMS) device mounted to, for example, a printed circuit board 28 enclosed within the housing 12 of the temperature measurement device 10.

In some examples, the temperature sensor 26 can be a commercially available electronic temperature sensor, as are known in the art, such as a thermistor. A thermistor can be configured to continuously or periodically monitor an electric signal, such as an analog signal representative of electric resistance of a portion of the thermistor, which changes with temperature. In some examples, as described in further detail hereinafter, an increase in core body temperature above about 38° C., or any other selected temperature, can be converted to a digital signal that is transmitted from the temperature measurement device 10 to a remote device. Other compatible small, micro-scale, or nano-scale temperature sensors or probes can be used as the temperature sensor 26.

The temperature measurement device 10 can further include a wireless transmitter 30 enclosed within the interior 12 of the housing 10. The wireless transmitter 30 can be a short-range transmitter that emits near-field radio frequency signals that are detected by a reader device in close proximity to the patient 2. In other examples, the wireless transmitter 30 can transmit data using other known data transmission protocols or standards including, for example, Bluetooth®, ZigBee, ultra-wide band (UWB) radio frequency, and others, as are known in the art. The wireless transmitter 30 can also transmit data via longer range transmission standards, such as via WiFi or over a cellular network (e.g., a 3G, 4G, 4G LTE, or 5G cellular network).

In some examples, the wireless transmitter 30 can be mounted to the printed circuit board 28 proximate to the temperature sensor 26. The wireless transmitter 30 can include a signal generating portion and one or more antennas embedded on or extending from the printed circuit hoard 28 for transmitting generated signals from the wireless transmitter 30. In some examples, the wireless transmitter 30 is configured for one-way data communication, where data is actively or passively transmitted from the temperature measurement device 10 to a remote electronic device. In other examples, the wireless transmitter 30 can include a transceiver or signal receiving portion that allows for two-way data communication. For example, temperature measurement information can be sent from the temperature measurement device 10 to a remote device and/or to a computer network. The temperature measurement device 10 can receive information, such as operating instructions, software upgrades, or other information from remote devices via two-way communication with the wireless transceiver.

As used herein, “active” data transmission refers to data that is transmitted by a powered wireless transmitter 30, such as a wireless transmitter comprising or connected to a battery. In such cases, the wireless transmitter 30 can be configured to transmit or emit, for example, radio frequency signal(s) that are detected by remote devices positioned proximate to and/or within range of the wireless transmitter 30.

In contrast, “passive” data transmission refers to a wireless transmission that is induced by another device, such as an external radio frequency reader or remote control device, meaning that the wireless transmitter 30 does not need to have an independent power source. For example, a wireless transmitter 30 that passively transmits data can comprise an induction coil. When the induction coil is in proximity to a source of radio frequency radiation or radio frequency emitter, a magnetic field is induced within the induction coil. A reader or remote control device can detect characteristics of and/or changes in the magnetic field to obtain measurements from the implanted sensor device. Once the external reader or remote control device is moved away from the patient or turned off, the passive device or transmitter stops generating detectable electromagnetic signals. In this way, an implanted sensor can provide temperature measurements on demand (i.e., when the source of radio frequency radiation and magnetic field detecting circuitry are provided in proximity to the implanted sensor) without relying on an implanted power source or battery.

In some examples, a remote electronic device in wireless communication with the temperature measurement device 10 is a portable electronic device, such as a smartphone, smartwatch, tablet computer, laptop computer, or similar portable computer device. The remote electronic device can also be a stationary computer device, such as a smart speaker device (e.g., Amazon Alexa or Google Home device) or desktop computer. The computer network connected to and/or in wireless communication with the temperature measurement device 10 can be a Local Area Network (LAN), such as a network of computer and computer devices at the patient's home connected by Ethernet or Wi-Fi connections. Alternatively, the computer network can be a larger computer network, such as a computer network administered by a medical facility, insurance network, or the Internet. In some examples, information transmitted from the temperature measurement device 10 can be uploaded to, for example, a dedicated website via a secure data connection. In such cases, patients, caregivers, medical professions, or other approved persons can view the patient's temperature information through, for example, an encrypted portal available through the website. In order to preserve patient privacy and integrity of medical data, the portal or website can be protected by a password, two-factor identification, and other security features, as are known in the art.

With continued reference to FIGS. 1A-1C, the temperature measurement device 10 can further include at least one computer device, controller, or processor 32. The computer device, controller, or processor 32 can be mounted on the printed circuit hoard 28 in proximity to the wireless transmitter 30 and temperature sensor 26, and can be enclosed within the interior 20 of the housing 12. The computer device, controller, or processor 32 can be electrically connected to the temperature sensor 26 and to the wireless transmitter 30 by, for example, conductive traces extending over a surface of the printed circuit board 28. The computer device, controller, or processor 32 can be configured to receive and process data representative of a core body temperature of the patient from the temperature sensor 26. The computer device, controller, or processor 32 can also be configured to cause the wireless transmitter 30 to transmit the processed data to a remote device or computer network.

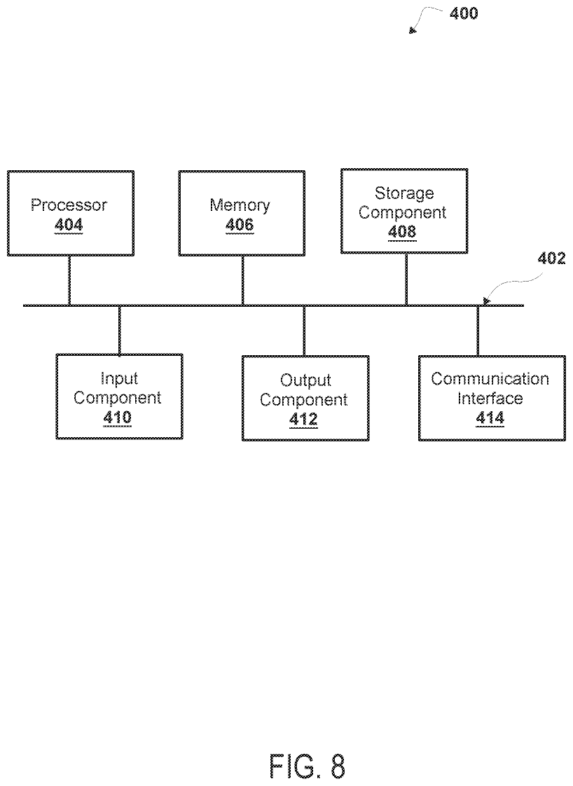

With reference to FIG. 8, an exemplary controller or computer device 400 that can be configured to receive and process information from the temperature sensor 26 can include, for example, a bus 402, a computer processor 404, memory 406, a storage component 408, an input component 410, an output component 412, and a communication interface 414. In some examples, the computer processor 404 can be implemented in hardware, software, or a combination of hardware and software. For example, the computer processor 404 may include a processor (e.g., a central processing unit (CPU), a graphics processing unit (CPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. The memory 406 can include random access memory (RAM), read-only memory (ROM), and/or another type of dynamic or static storage device (e.g., flash memory, magnetic memory, optical memory, etc.) that stores information and/or instructions for use by computer processor 404.

The controller or computer device 400 can be configured to perform one or more processes described herein. The controller or computer device 400 can perform these processes based on execution of software instructions by the processor 404 (e.g., a central processing unit (CPU), a graphics processing unit (GPU), etc.). The software instructions can be stored, for example, by a computer-readable medium, such as the memory 406 and/or the storage component 408. A computer-readable medium (e.g., a non-transitory computer-readable medium) is defined herein as a non-transitory memory device. A non-transitory memory device includes memory space located inside of a single physical storage device or memory space spread across multiple physical storage devices. When executed, software instructions stored in the memory 406 and/or the storage component 408 may cause the computer processor 404 to perform one or more processes described herein. Additionally or alternatively, hardwired circuitry may be used in place of or in combination with software instructions to perform one or more processes described herein. The memory 406 and/or storage component 408 may include data storage or one or more data structures (e.g., a database, etc.). The controller or computer device 400 may be capable of receiving information from, storing information in, communicating information to, or searching information stored in the data storage or one or more data structures in the memory 406 and/or the storage component 408.

With continued reference to FIGS. 1A-1C, in some examples, the receiving and processing of data from the temperature sensor 26 by the computer device, controller, or processor 32 can include, for example, filtering a signal received from the temperature sensor 26 to remove signal noise or to otherwise prepare the signal for wireless data transmission, Processing the signal can also include, for example, converting an analog signal received from the temperature sensor 26 to a digital signal. In some examples, processing the signal can also include analyzing the signal to determine a core body temperature value for the patient's core body temperature. In such cases, the determined core body temperature value can be wirelessly transmitted to the remote device. Alternatively, signal analysis to determine the core body temperature value can be performed on the remote device based on analog or digital signals transmitted from the temperature measurement device 10 via the wireless transmitter 30.

In some examples, the computer device, controller, or processor 32 can be configured to cause the wireless transmitter 30 to continuously stream temperature measurement data from the temperature measurement device 10 to the remote device or computer network. Alternatively, the computer device, controller, or processor 32 can be configured to cause the wireless transmitter 30 to transmit data on a periodic basis, such as at a predetermined interval (e.g., every 10 minutes, 30 minutes, or 60 minutes). Further, in some examples, the computer device, controller, or processor 32 of the temperature measurement device 10 can be configured to monitor temperature measurement data to determine when the patient has a fever (e.g., a core body temperature in excess of about 33° C.) or a significant fever (e.g., a core body temperature in excess of 39° C. or 40° C.). When a fever or significant fever is detected, the computer device, controller, or processor 32 can be configured to cause the wireless transmitter 30 to transmit a notification, alert, or alarm to the remote device or computer network to alert the patient or caregiver.

In some examples, as previously described, the temperature measurement device 10 also includes a battery 34 for providing power for the temperature sensor 26, the processor 32, and/or the wireless transmitter 30.. For example, the battery 34 can be positioned and sealed within the interior 20 of the housing 12 and electrically connected to electronic components of the device 10 enclosed within the housing 12. The battery 34 can be, for example, a lithium ion battery, as used for pacemakers and similar medical devices, or any other battery of sufficient lifespan and size to provide power for electronic devices for the entire useful life of the temperature measurement device 10 and venous access port 112. Since implantable venous access ports 112 are often only used for several months, the lifespan of the battery 34 may only need to be several months of continuous or substantially continuous use.

### Temperature Monitoring System

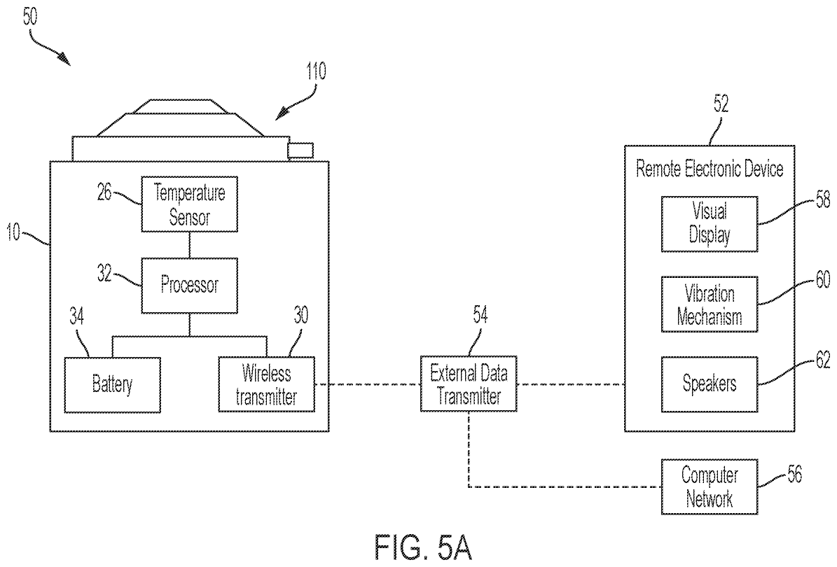

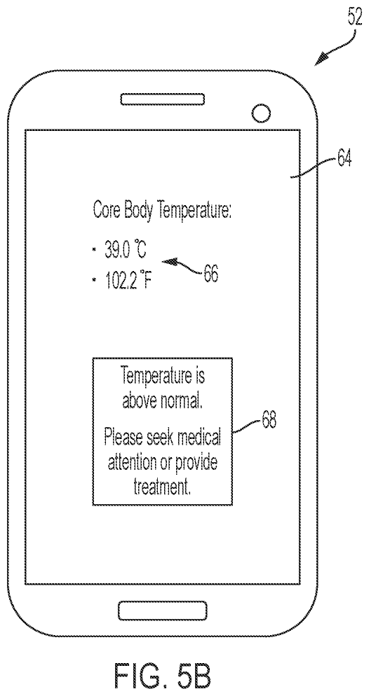

The temperature measurement device 10 of the present disclosure can be integrated into a temperature monitoring system 50 configured to provide temperature information, as well as a notification, alarm, or alert when a fever is detected, to the patient and caregivers. As shown in FIG. 5A, the temperature monitoring system 50 includes the temperature measurement device 10. As previously described, the temperature measurement device 10 is implanted in the patient along with the implantable medical device 110, such as the venous access port 112.

The system 50 further includes the remote electronic device 52, which is in direct or indirect wireless communication with the temperature measurement device 10. In particular, the remote electronic device 52 receives processed data and information about the core body temperature of the patient from the temperature measuring device 10. As previously described, the remote electronic device 52 can be a portable electronic device, such as a smartphone, smartwatch, tablet computer, laptop computer, or similar portable computer device. The remote device 52 can also be a stationary computer device, such as a smart speaker device (e.g., Amazon Alexa or Google Home), smart television, or desktop computer,

In some examples, the remote electronic device 52 is in “direct wireless communication” with the wireless transmitter 30 of the temperature measurement device 10. For example, the wireless transmitter 30 of the temperature measurement device 10 can transmit data and information using a conventional and widely accepted data short-range transmission standard, such as Bluetooth®, which can be received by the remote electronic device 52 or by a computer network 56. As used herein, “direct wireless communication” means that signals are transmitted directly from a first device to a second device, without being received, processed, and/or retransmitted by an intermediary device.

Alternatively, as shown in FIG. 5A, the temperature monitoring system 50 can include an intermediary device, such as an external data transmitter 54. The external data transmitter 54 can be positioned in proximity to the implanted temperature measurement device 10 and can be configured to receive signals from the wireless transmitter 30 of the temperature measurement device 10 and to retransmit the received signals. For example, the external data transmitter 54 can be an electronic device worn by the patient 2 at a position proximate to the implanted temperature measurement device 10 with, for example, a holster, belt, vest, backpack, armband, pocket, or some other wearable support structure, depending upon the location where the temperature measurement device 10 is implanted.

In some examples, the external data transmitter 54 can be configured to receive signal(s), such as near infra-red signals, from the wireless transmitter 30 of the temperature monitoring device 10. The external data transmitter 54 can be configured to process the signals and to retransmit the signals in a different data transmission standard that can be received by other electronic device. For example, the external transmitter 54 can be configured to receive near infra-red signals from the wireless transmitter 30 of the temperature measurement device 10 and to retransmit the signals in another data transmission standard to the remote electronic device 52 by a short-range data transmission standard (e.g., Bluetooth® or ZigBee). The external transmitter 54 may also transmit signals to a computer network 56, such as a Local Area Network, computer network implemented by a medical facility or healthcare company, or to the Internet. For example, the external transmitter 54 can include a WiFi or cellular transmitter for sending signals to a WiFi or cellular network. Received data could then be downloaded from the computer network 56 by remote electronic devices 52 used by the patient 2, caregivers, or other individuals with appropriate access. For example, as previously described, the patient 2 or caregiver could access temperature information on a dedicated website via a secure data connection.

In some examples, the external data transmitter 54 includes a radio frequency emitter configured to provide a radio frequency signal to a passive wireless transmitter 30, such as an induction coil. As previously described, when exposed to the radio frequency signal from the radio frequency emitter, a magnetic field is created with the induction coil. Electronic circuitry of the external data transmitter 54 can detect parameters of the created magnetic field, which correspond to a temperature sensed by the temperature sensor 26. As in previous examples, the external data transmitter 54 can be configured to transmit information about the sensed temperature to the remote electronic device 52 and/or to a computer network 56,

Once signals and/or processed data is received by the remote electronic device 52, the remote electronic device 52 is configured to provide the indications representative of the received information or data to users (e.g., to the patient or caregiver). The feedback can be, for example, visual feedback shown on a visual display 58 of the remote electronic device 52, haptic feedback output by a vibration mechanism 60 of the remote electronic device 52, and/or audio feedback output from speakers 62 of the remote electronic device 52.

An example of a user interface screen 64 displayed on the visual display 58 with information about the patient's temperature is shown in FIG. 5B. As shown in FIG. 5B, feedback on the user interface screen 64 can include displaying a numeric value 66 for the patient's core body temperature detected by the temperature sensor 26. The feedback can also include indications showing whether the patient's core body temperature is normal (e.g., at or below 38° C.), abnormal (e.g., above 38° C.), or substantially abnormal (above 39° C. or 40° C.). For example, numerical values 66 can include highlighting or be presented in different colors to indicate that the patient has a fever (e.g., numerical values highlighted in yellow) or a substantial fever (e.g., numerical values highlighted in red). Also, the remote electronic device 52 can provide haptic or audio feedback to indicate that the patient has a fever. For example, the remote electronic device 52 may vibrate or emit a warning sound when a fever is detected. As previously described, in some examples, temperature values shown on the remote electronic device can be updated in real time or substantially continuously from data streamed from the temperature measurement device 10. In other examples, the displayed temperature values can be updated periodically, such as at predetermined intervals. In other examples, the remote electronic device 52 can be configured to provide feedback only when a fever is detected. For example, the remote electronic device 52 may provide a text and/or visual notification 68 on the display screen 58 and/or may provide haptic feedback (e.g., aggressive vibrations) and audio warnings to alert the patient 2 or caregiver the patient 2 has a fever and to seek medical attention.

### Core Temperature Monitoring Method

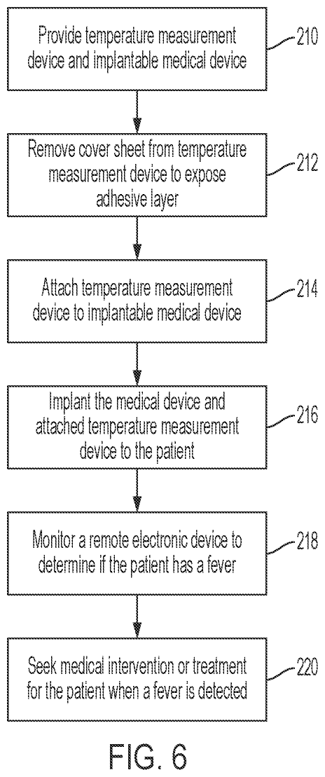

FIG. 6 is a flowchart showing exemplary steps of a method of monitoring a core body temperature of a non-communicative patient. The non-communicative patient can be, for example, a child, an elderly patient, and/or a patient with dementia, Alzheimer's, or any other patient who has difficulty expressing to a caregiver when they feel or may have a fever. Temperature monitoring can be especially important for non-communicative patients so that fevers are quickly identified and appropriate medical intervention is sought. In some examples, the patient is an immuno-comprised patient, such as a cancer patient, who is susceptible to infections, such as blood infections. Further, the patient is generally a patient that requires a long-term implanted medical device, such as a venous access port and venous catheter, which can be a source for blood infections. As previously described, such medical ports are commonly used for providing infusions for drugs, blood, or nutrition to patients, such as patients undergoing chemotherapy treatment or treatment with other cancer-fighting medicines, IV treatments (e.g., intravenous infusion of antibiotics and nutrition), and/or patients undergoing hemodialysis treatment for kidney failure.

At step 210, the method includes providing a temperature measurement device 10 and an implantable medical device 110, such as a venous access port 112, to be implanted for the non-communicative patient. At step 212, the method next includes removing or peeling away the cover sheet 24 from the temperature measurement device 10 to expose the adhesive layer 22 of the temperature measurement device 10. At step 214, the method further includes attaching the temperature measuring device 10 to the implantable medical device 110. As previously described, the temperature measurement device 10 and the implantable medical device 110 are generally similar in size and shape. For example, the top 14 of the temperature measurement device 10 can be about the same size and shape as the flat bottom 118 of the :implantable medical device 110. In such cases, the exposed adhesive layer 22 on the top 14 of the temperature measurement device 10 can be pressed against the flat bottom 118 of the implantable medical device 110, thereby securing the temperature measurement device 10 to the implantable medical device 110.

At step 216, the method further includes implanting the implantable medical device 110, such as the venous access port 112, and the attached temperature measurement device 10 to the patient. In some examples, implanting the devices 10, 110 to the patient includes, for example, making an incision over a front portion of the patient's chest and inserting the devices 10, 110 into the subcutaneous space between the patient's skin and the ribs and/or muscle tissue. The devices 10, 110 can be secured in place by sutures or other surgical techniques used for implanting medical ports, pacemakers, and other subcutaneously implanted medical devices, as are known in the art. Implanting the medical device 10 can also include positioning the catheter 12.8 within a vein of the patient and/or connecting the catheter 128 to the port 112 of the medical device 110. As previously described, FIG. 4 shows the temperature measurement device 10 and venous port 112 implanted in a subcutaneous space above the chest 4 of the patient 2. It is also understood that the devices 10, 110 can also be implanted in other body locations, such as the upper arm or leg, within the scope of the present disclosure, depending upon the type of treatment being provided to the patient.

At step 218, the method further includes monitoring a remote electronic device 52, such as a portable electronic device (e.g., smartphone, computer tablet, or laptop computer), in wireless communication with the temperature measurement device 10 to determine if the patient has a fever. For example, monitoring the remote electronic device 52 can include periodically viewing the visual display 58 of the remote electronic device 52 to read the displayed temperature value. As previously discussed, the remote electronic device 52 may also be configured to emit a notification, alert, or alarm when the patient has a fever (a core body temperature in excess of 38° C.) or a substantial fever (e.g., a core body temperature in excess of, for example, 39° C. or 40° C.).

At step 220, monitoring the patient can further include seeking appropriate medical intervention when a fever is detected, For example, when the patient's core body temperature indicates that the patient has a substantial fever (e.g., a core body temperature in excess of, for example, 39° C. or 40° C.), the patient and/or caregiver may alert a medical professional and/or the patient can be taken to a medical facility, such as an emergency room. As previously discussed, for immuno-compromised patients, it can be important that the patient receives medical treatment shortly after a fever is detected, desirably within about 60 minutes, 120 minutes, or 180 minutes of detecting the fever.

### Integrated Access Port and Temperature Measurement Device

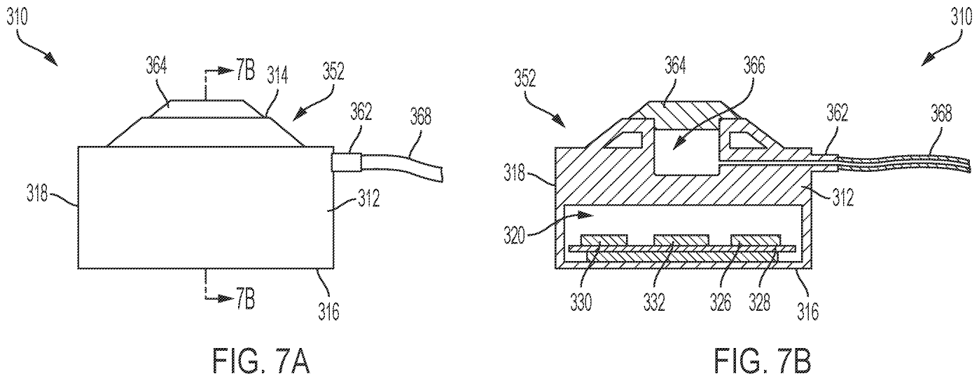

FIGS. 7A and 7B show an integrated device 310 including electronic circuitry for measuring and wirelessly transmitting information about a patient's core body temperature integrated with an implantable medical device, such as a venous access port 352. The integrated device 310 includes a housing 312. As in previous examples, the housing 312 can be made from a biocompatible rigid plastic, such as polyurethane or high density polyethylene. The housing 312 includes an open top 314, a bottom 316, at least one sidewall 318 extending between the top 314 and the bottom 316 of the housing 312. The housing 312 also includes a catheter port 362 extending from the sidewall 318 of the housing 312. The housing 312 defines a fluid chamber 366 (shown in FIG. 7B) in fluid communication with the catheter port 362. In addition, the housing 312 includes or defines a separate interior 320 (shown in FIG. 7B) sized to contain electrical components of the integrated device 310.

The integrated device 310 further includes a pierceable, self-sealing septum 364 over the open top 314 of the housing 312. As in previous examples, the septum 364 can be formed from a self-sealing elastomeric material, such as silicone or synthetic rubber. The septum 364 is configured to allow for injection of liquids into the fluid chamber 366 of the housing 312 by piercing the septum 364 and injecting liquid through the septum 364 into the chamber 366.

The integrated temperature measurement device 310 can also include a venous catheter 368 connected to and extending from the catheter port 362. The venous catheter 368 can be configured to extend from the catheter port 362 into a vein of the patient. The venous catheter 368 is fluidly connected to the fluid chamber 366 so that liquids injected into the chamber 366 move through the venous catheter 368 and to the vein of the patient.

The integrated device 310 further includes the electronic circuitry for detecting the core body temperature of the patient and for transmitting data, signals, and/or measured values from the temperature measurement device 310 to the remote electronic device 52 (shown in FIG. 5A). For example. the integrated temperature measurement device 310 can include a temperature sensor 326 connected to the housing 312 and/or enclosed within the interior 320 of the housing 312. The temperature sensor 326 and associated electronic components can be mounted to a printed circuit board 328 enclosed within the interior 320 of the housing 312. The temperature measurement device 310 can also include a wireless transmitter 330 and a computer device, controller, or processor 332, which can also be mounted to and electrically connected together on the printed circuit hoard 328. As in previous examples, the computer device, controller, or processor 332 is electrically connected to the temperature sensor 326 and to the wireless transmitter 330. Further, the computer device, controller, or processor 332 is configured to receive and process data representative of a core body temperature of the patient from the temperature sensor 326. The computer device, controller, or processor 332 is also configured to cause the wireless transmitter 330 to transmit the processed data to the remote device 52 or computer network 56 (shown in FIG. 5A).

The integrated temperature measurement device 310 of FIGS. 7A and 7B can be implanted and used in the same manner as previously described examples. For example, the integrated device 310 can be implanted into the subcutaneous space through an incision over the patient's chest and/or proximate to the patient's clavicle. Once implanted, the temperature measurement device 310 wirelessly transmits core body temperature data to the remote electronic device 52, which can be monitored by the patient and/or by the patient's caregiver to determine whether the patient has a fever. When a fever is detected, the patient can seek appropriate medical intervention.

Non-limiting aspects or embodiments of the present invention now be described in the following numbered clauses:

Clause 1: A temperature measurement device configured to be attached to an implantable medical device for measuring a core body temperature of a patient, the temperature measurement device comprising: a housing comprising a top, a bottom, and at least one sidewall extending between the top and the bottom of the housing enclosing an interior; a temperature sensor connected to the housing and/or enclosed within the interior of the housing; a wireless transmitter enclosed within the interior of the housing; and at least one processor enclosed within the interior of the housing electrically connected to the temperature sensor and to the wireless transmitter configured to: receive and process data representative of a core body temperature of the patient from the temperature sensor; and cause the wireless transmitter to transmit the processed data to a remote device or computer network,

Clause 2: The temperature measurement device of clause 1, wherein a shape and size of at least one of the top or the bottom of the housing substantially matches a size and shape of a surface of the implantable medical device to which the housing is to be attached.

Clause 3: The temperature measurement device of clause 1 or clause 2, wherein a diameter of at least one of the top or the bottom of the housing is substantially equivalent to a diameter of a top or a bottom surface of the implantable medical device.

Clause 4: The temperature measurement device of any of clauses 1-3, wherein the housing comprises a waterproof and sealed housing.

Clause 5: The temperature measurement device of any of clauses 1-4, wherein the housing comprises a biocompatible rigid plastic material.

Clause 6: The temperature measurement device of clause 5, wherein the biocompatible rigid plastic material comprises polyurethane or high-density polyethylene.

Clause 7: The temperature measurement device of any of clauses 1-6, wherein the temperature sensor comprises an electric thermistor that measures an analog electric signal representative of a temperature of an environment around the thermistor.

Clause 8: The temperature measurement device of any of clauses 7 wherein the wireless transmitter comprises a near-field radio frequency transmitter.

Clause 9: The temperature measurement device of any of clauses 1-8, wherein the at least one processor causes the wireless transmitter to transmit the received and processed data to the remote device or computer network at predetermined periodic intervals.

Clause 10: The temperature measurement device of any of clauses 1-9, wherein the at least one processor is further configured to: periodically analyze the processed data to determine the core body temperature of the patient, and cause the wireless transmitter to transmit a notification to the remote device or computer network when the patient has a fever (e.g., a core body temperature above 38° C.).

Clause 11: The temperature measurement device of any of clauses 1-10, further comprising a battery (e.g., a lithium ion battery) in the interior of the housing for providing power for the temperature sensor, the at least one processor, and the wireless transmitter.

Clause 12: The temperature measurement device of clause 11, wherein the battery is sealed within the housing.

Clause 13: The temperature measurement device of any of clauses 1-12, wherein the wireless transmitter passively transmits data to the remote device when the wireless transmitter is exposed to a radio frequency emitter,

Clause 14: The temperature measurement device of any of clauses 1-13, wherein the implantable medical device comprises an implantable cardiovascular device.

Clause 15: The temperature measurement device of any of clauses 1-14, wherein the implantable medical device comprises an implantable venous access port.

Clause 16: The temperature measurement device of clause 15, wherein the implantable venous access port comprises: a housing comprising an open top, a closed bottom, a sidewall extending between the top and the bottom of the housing, and a catheter port extending from the sidewall of the housing; a pierceable, self-sealing septum over the open top of the housing configured for injection of liquids into a fluid chamber enclosed by the housing; and a venous catheter connected to and extending from the catheter port, wherein the venous catheter is configured to extend from the catheter port into a vein of the patient and is fluidly connected to a fluid chamber enclosed by the housing so that fluid injected into the chamber passes into the venous catheter and to the vein of the patient.

Clause 17: The temperature measurement device of any of clauses 1-16, wherein the implantable medical device is configured to be implanted in a subcutaneous space over the patient's chest below the patient's skin and above muscle tissue and/or ribs of the patient.

Clause 18: The temperature measurement device of any of clauses 1-17, wherein the housing, the temperature sensor, and electrics of the temperature measurement device are :MRI compatible.

Clause 19: The temperature measurement device of any of clauses 1-18, wherein the wireless transmitter comprises a near-field radio frequency transmitter and the remote device comprises at least one external data transmitter configured to be worn by the patient and to wirelessly transmit data received from the temperature measurement device to a portable electronic device.

Clause 20: The temperature measurement device of any of clauses 1-19, further comprising an adhesive layer over at least one of the top or the bottom of the housing for attaching the housing to a surface of the implantable medical device.

Clause 21: The temperature measurement device of clause 20, further comprising a removable cover sheet over the adhesive layer configured to be removed from the adhesive layer so that the temperature measurement device can be adhered to the implantable medical device.

Clause 22: The temperature measurement device of any of clauses 1-21, wherein the temperature measurement device is integrally formed and enclosed within a common housing with the implantable medical device.

Clause 23: An implantable assembly comprising: the temperature measurement device of any of clauses 1-19; and a venous access port connected to the temperature measurement device, wherein the venous access port comprises: a housing comprising an open top, a closed bottom, a sidewall extending between the top and the bottom of the housing, and a catheter port extending from the sidewall of the housing; a pierceable, self-sealing septum over the open top of the housing configured for injection of liquids into a fluid chamber enclosed by the housing; and a venous catheter connected to and extending from the catheter port, wherein the venous catheter is configured to extend from the catheter port into a vein of the patient and is fluidly connected to a fluid chamber enclosed by the housing for moving fluid injected into the fluid chamber through the venous catheter and to the vein of the patient.

Clause 24: The assembly of clause 23, further comprising an adhesive layer over at least one of the top or the bottom of the housing of the temperature measurement device that attaches the housing of the temperature measurement device to the closed bottom of the housing of the venous access port,

Clause 25: The assembly of clause 23, wherein the temperature measurement device is integrally formed and enclosed within a common housing with the venous access port.

Clause 26: An implantable medical device, comprising: a housing comprising an open top, a bottom, and at least one sidewall extending between the top and the bottom of the housing, and a catheter port extending from the sidewall of the housing, wherein the housing defines a fluid chamber in fluid communication with the catheter port and a separate interior; a pierceable, self-sealing septum over the open top of the housing configured for injection of liquids into the fluid chamber of the housing; a venous catheter connected to and extending from the catheter port, wherein the venous catheter is configured to extend from the catheter port into a vein of the patient and is fluidly connected to a fluid chamber of the housing so that fluid injected into the fluid chamber passes through the venous catheter and to the vein of the patient; a temperature sensor connected to and/or enclosed within the interior of the housing; a wireless transmitter enclosed within the interior of the housing; and at least one processor enclosed within the interior of the housing electrically connected to the temperature sensor and to the wireless transmitter configured to: receive and process data representative of a core body temperature of the patient from the temperature sensor; and cause the wireless transmitter to transmit the processed data to a remote device or computer network.

Clause 27: The implantable medical device of clause 26, wherein the wireless transmitter comprises a near-field radio frequency transmitter.

Clause 28: The implantable medical device of clause 26 or clause 27, wherein the at least one processor causes the wireless transmitter to transmit the received and processed data to the remote device or computer network at predetermined periodic intervals.

Clause 29: The implantable medical device of any of clauses 26-28, wherein the at least one processor is further configured to: periodically analyze the processed data to determine the core body temperature of the patient, and cause the wireless transmitter to transmit a notification to the remote device or computer network when the patient has a fever (e.g., a core body temperature in excess of 38° C.)

Clause 30: The implantable medical device of any of clauses 26-29, further comprising a battery (e.g., a lithium ion battery) in the interior of the housing for providing power for the temperature sensor, the at least one processor, and the wireless transmitter.

Clause 31: The implantable medical device of any of clauses 26-30, wherein the wireless transmitter passively transmits data to the remote electronic device when the wireless transmitter is exposed to a radio frequency emitter.

Clause 32: A temperature monitoring system, comprising: the assembly or device of any of clauses 23-31; and at least one remote electronic device configured to directly or indirectly receive processed data from the temperature measurement device and output information about a core body temperature of a patient determined based on the received data,

Clause 33: The temperature monitoring system of clause 32, further comprising at least one external data transmitter configured to receive signals comprising the processed data from the wireless transmitter of the temperature measurement device and to retransmit the received signals to the at least one remote electronic device.

Clause 34: The temperature monitoring system of clause 32 or clause 33, wherein the remote electronic device comprises at least one of a smartphone, smartwatch, computer tablet, smart speaker, laptop, or personal computer.

Clause 35: The temperature monitoring system of any of clauses 32-34, wherein the at least one remote electronic device is configured to provide an alarm when the patient has a fever (e.g., a measured core body temperature above about 38° C., 39° C., or 40° C.).

Clause 36: The temperature monitoring system of any of clauses 32-35, wherein the alarm is output by a visual display, haptic feedback device, and/or speakers of the remote electronic device.

Clause 37: A method of monitoring a core body temperature of a non-communicative patient, comprising: attaching the temperature measurement device of any of clauses 1-19 to an implantable medical device; implanting the :implantable medical device and attached temperature measurement device to the patient; and monitoring a remote electronic device in wireless communication with the temperature measurement device to determine if the patient has a fever (e.g., a core body temperature above about 38° C., 39° C., or 40° C.), wherein the remote electronic device is configured to receive the processed data from the temperature measurement device; and provide an indication of the core body temperature of the patient via the remote electronic device.

Clause 38: A method of monitoring a core body temperature of a non-communicative patient, comprising: implanting the assembly or device of any one of clauses 23-31 to the patient; and monitoring a remote electronic device in wireless communication with the temperature measurement device to determine if the patient has a fever (e.g., a core body temperature above about 38° C., 39° C., or 40° C.), wherein the remote electronic device is configured to: receive the processed data from the temperature measurement device; and provide an indication of the core body temperature of the patient via the remote electronic device.

Clause 39: The method of clause 37 or 38, wherein the provided indication comprises a visual notification on a display screen of the remote electronic device, a haptic notification output by a haptic vibrator of the remote electronic device, and/or an audible notification output by speakers of the portable remote device.

Clause 40: The method of any of clauses 37-39, wherein the non-communicative patient is a child, an elderly patient, and/or a patient with dementia or Alzheimer's disease.

Clause 41: The method of any of clauses 37-40, wherein attaching the temperature measurement device to the implantable medical device comprises removing a cover sheet from the temperature measurement device to expose an adhesive layer of the temperature measurement device, and attaching the temperature measurement device to the implantable medical device with the exposed adhesive layer.

Clause 42: The method of any of clauses 37-41, wherein device or assembly comprises an implantable cardiovascular device.

Clause 43: The method of any of clauses 37-42, wherein the device or assembly comprises a venous access port.

Clause 44: The method of clause 43, wherein the venous access port comprises: a housing comprising an open top, a closed bottom, a sidewall extending between the top and the bottom of the housing, and a catheter port extending from the sidewall of the housing; a pierceable, self-sealing septum over the open top of the housing configured for injection of liquids into a fluid chamber enclosed by the housing; and a venous catheter connected to and extending from the catheter port, wherein the venous catheter is configured to extend from the catheter port into a vein of the patient and is fluidly connected to a fluid chamber enclosed by the housing for moving fluid injected into the fluid chamber through the venous catheter and to the vein of the patient.

Clause 45: The method of clause 43 or clause 44, wherein implanting the device or assembly comprises implanting the device or assembly into a subcutaneous space between the skin and muscle tissue of the patient.

Clause 46: The method of any of clauses 43-45, wherein the venous access port is implanted to provide at least one of a drug infusion, blood infusion, or nutrition to the patient.

Clause 47: The method of clause 46, wherein the patient is undergoing chemotherapy treatment or treatment with other cancer-fighting medicines, IV treatments (e.g., intravenous infusion of antibiotics and nutrition), and/or hemodialysis treatment for kidney failure

Clause 48: Use of the device or assembly of any of clauses 1-31 for monitoring a core body temperature of a non-communicative patient, wherein the patient is optionally undergoing at least one of chemotherapy treatment or treatment with other cancer-fighting medicines, IV treatments (e.g., intravenous infusion of antibiotics and nutrition), and/or hemodialysis treatment for kidney failure.

Clause 49: Use of the temperature measurement device or assembly of any of clauses 1-31 for monitoring a core body temperature of a patient, wherein the patient is a child, an elderly patient, and/or a patient with dementia or Alzheimer's disease.

1. A temperature measurement device configured to be attached to an implantable medical device for measuring a core body temperature of a patient, the temperature measurement device comprising:
a housing comprising a top, a bottom, and at least one sidewall extending between the top and the bottom of the housing enclosing an interior;
a temperature sensor connected to the housing and/or enclosed within the interior of the housing;
a wireless transmitter enclosed within the interior of the housing; and
at least one processor enclosed within the interior of the housing electrically connected to the temperature sensor and to the wireless transmitter configured to:
receive and process data representative of a core body temperature of the patient from the temperature sensor; and
cause the wireless transmitter to transmit the processed data to a remote device or computer network.

2. The temperature measurement device of claim 1, wherein a shape and size of at least one of the top or the bottom of the housing substantially matches a size and shape of a surface of the implantable medical device to which the housing is to be attached.

3. The temperature measurement device of claim 1, wherein a diameter of at least one of the top or the bottom of the housing is substantially equivalent to a diameter of a top or a bottom surface of the implantable medical device.

4. The temperature measurement device of claim 1, wherein the housing comprises a waterproof and sealed housing

5. (canceled)

6. (canceled)

7. The temperature measurement device of claim 1, wherein the temperature sensor comprises an electric thermistor that measures an analog electric signal representative of a temperature of an environment around the thermistor.

8. The temperature measurement device of claim 1, wherein the wireless transmitter comprises a near-field radio frequency transmitter, and/or
wherein the wireless transmitter passively transmits data to the remote device when the wireless transmitter is exposed to a radio frequency emitter.

9. The temperature measurement device of claim 1, wherein the at least one processor causes the wireless transmitter to transmit the received and processed data to the remote device or computer network at predetermined periodic intervals.

10. The temperature measurement device of claim 1, wherein the at least one processor is further configured to:
periodically analyze the processed data to determine the core body temperature of the patient, and
cause the wireless transmitter to transmit a notification to the remote device or computer network when the patient has a fever.

11. The temperature measurement device of claim 1, further comprising a battery in the interior of the housing for providing power for the temperature sensor, the at least one processor, and the wireless transmitter,

12. (canceled)

13. (canceled)

14. The temperature measurement device of claim 1, wherein the implantable medical device comprises an implantable cardiovascular device.

15. The temperature measurement device of claim 1, wherein the implantable medical device comprises an implantable venous access port.

16. The temperature measurement device of claim 15, wherein the implantable venous access port comprises:
a housing comprising an open top, a closed bottom, a sidewall extending between the top and the bottom of the housing, and a catheter port extending from the sidewall of the housing;
a pierceable, self-sealing septum over the open top of the housing configured for injection of liquids into a fluid chamber enclosed by the housing; and
a venous catheter connected to and extending from the catheter port, wherein the venous catheter is configured to extend from the catheter port into a vein of the patient and is fluidly connected to a fluid chamber enclosed by the housing so that fluid injected into the chamber passes into the venous catheter and to the vein of the patient.

17. The temperature measurement device of claim 1, wherein the implantable medical device is configured to be implanted in a subcutaneous space over the patient's chest below the patient's skin and above muscle tissue and/or ribs of the patient.

18. (canceled)

19. The temperature measurement device of claim 1, wherein the wireless transmitter comprises a near-field radio frequency transmitter and the remote device comprises at least one external data transmitter configured to be worn by the patient and to wirelessly transmit data received from the temperature measurement device to a portable electronic device.

20. The temperature measurement device of claim 1, further comprising an adhesive layer over at least one of the top or the bottom of the housing for attaching the housing to a surface of the implantable medical device, and optionally, a removable cover sheet over the adhesive layer configured to be removed from the adhesive layer so that the temperature measurement device can be adhered to the implantable medical device.

21. (canceled)

22. (canceled)

23. An implantable assembly comprising:
the temperature measurement device of claim 1; and
a venous access port connected to the temperature measurement device, wherein the venous access port comprises:
a housing comprising an open top, a closed bottom, a sidewall extending between the top and the bottom of the housing, and a catheter port extending from the sidewall of the housing;
a pierceable, self-sealing septum over the open top of the housing configured for injection of liquids into a fluid chamber enclosed by the housing; and
a venous catheter connected to and extending from the catheter port, wherein the venous catheter is configured to extend from the catheter port into a vein of the patient and is fluidly connected to a fluid chamber enclosed by the housing for moving fluid injected into the fluid chamber through the venous catheter and to the vein of the patient.

24. (canceled)

25. (canceled)

26. An implantable medical device, comprising:
a housing comprising an open top, a bottom, and at least one sidewall extending between the top and the bottom of the housing, and a catheter port extending from the sidewall of the housing, wherein the housing defines a fluid chamber in fluid communication with the catheter port and a separate interior;
a pierceable, self-sealing septum over the open top of the housing configured for injection of liquids into the fluid chamber of the housing;
a venous catheter connected to and extending from the catheter port, wherein the venous catheter is configured to extend from the catheter port into a vein of the patient and is fluidly connected to a fluid chamber of the housing so that fluid injected into the fluid chamber passes through the venous catheter and to the vein of the patient;
a temperature sensor connected to and/or enclosed within the interior of the housing;
a wireless transmitter enclosed within the interior of the housing; and
at least one processor enclosed within the interior of the housing electrically connected to the temperature sensor and to the wireless transmitter configured to:
receive and process data representative of a core body temperature of the patient from the temperature sensor; and
cause the wireless transmitter to transmit the processed data to a remote device or computer network.

27-31. (canceled)

32. A temperature monitoring system, comprising:
the implantable assembly of claim 23; and
at least one remote electronic device configured to directly or indirectly receive processed data from the temperature measurement device and output information about a core body temperature of a patient determined based on the received data.

33-36. (canceled)

37. A method of monitoring a core body temperature of a non-communicative patient, comprising:
attaching the temperature measurement device of claim 1 to an implantable medical device;
implanting the implantable medical device and attached temperature measurement device to the patient; and
monitoring a remote electronic device in wireless communication with the temperature measurement device to determine if the patient has a fever
wherein the remote electronic device is configured to:
receive the processed data from the temperature measurement device; and
provide an indication of the core body temperature of the patient via the remote electronic device.

38. (canceled)

39. (canceled)

40. The method of claim 37, wherein the non-communicative patient is a child, an elderly patient, and/or a patient with dementia or Alzheimer's disease.

41-49. (canceled)
